(19) United States
(12) Patent Application Publication
(10) Pub. No.: US 2008/0071173 A1
(43) Pub. Date: Mar. 20, 2008

(54) Visualizing Formation of Ablation Lesions

(76) Inventor: William N. Aldrich, Napa, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/532,814

(22) Filed: Sep. 18, 2006

(51) Int. Cl. A61B 8/00

(52) U.S. Cl. 600/439

(57) ABSTRACT

Systems and methods for visualizing formation of ablation lesions are provided therein. The systems and methods achieve this by alternately performing ultrasound imaging for a short time interval and performing ablation for a short time interval such that the ultrasound imaging appears to show the ablation occurring in real time.

### FIELD OF THE INVENTION

The field of the invention relates generally to ablation, and more particularly to visualizing formation of ablation lesions.

### BACKGROUND INFORMATION

Ablation is used to treat various medical conditions by destroying selected tissue in a patient's body. For example, ablation is used to treat cardiac arrhythmia by destroying diseased heart tissue responsible for abnormal electrical pathways in the heart. This is typically done by guiding a catheter or probe with a radio frequency (RF) transducer into the heart, and positioning the transducer near the tissue to be ablated. Once positioned, the transducer is excited to apply RF energy to the tissue to be ablated. The RF energy causes the tissue to heat up and die forming an ablation lesion. Ablation can also be used to treat obesity by ablating the vagal nerve. Ablation of the vagal nerve is described in U.S. patent application Ser. No. 10/389,236, titled “Methods and Apparatus for Treatment of Obesity,” filed Mar. 14, 2003.

During an ablation procedure, it is important to ablate the desired tissue while avoiding ablation of surrounding healthy tissue. Accidental ablation of healthy tissue can lead to serious injury and even death. Ultrasound imaging has been used to visualize ablated tissue after an ablation procedure to access the effectiveness of the ablation. However, this does not allow a clinician to observe the formation of ablation lesions during the ablation procedure. Further, ultrasound imaging may not be performed simultaneously with ablation to visualize the formation of ablation lesions because the ablation energy may interfere with or overload the ultrasound imaging, which may result in whiteout of the ultrasound images.

Therefore, there is a need for systems and methods that visualize the formation of ablation lesions. This would allow a clinician to quickly detect ablation in an undesired region and to immediately stop the ablation to prevent damage to healthy tissue.

### SUMMARY

Systems and methods for visualizing formation of ablation lesions are provided therein. The systems and methods achieve this by alternately performing ultrasound imaging for a short time interval and performing ablation for a short time interval such that the ultrasound imaging appears to show the ablation occurring in real time.

A system according to an embodiment comprises a controller, an ultrasound system and an ablation generator. The controller controls ultrasound image acquisition by the ultrasound system and controls power to the ablation generator. During an ablation procedure, the controller alternately triggers the ultrasound imaging system to acquire an ultrasound image with the ablation generator powered off and powers on the ablation generator for a short time interval with the ultrasound imaging off. Because the system alternates between the ultrasound imaging and the ablation at a fast rate, the ultrasound system appears to show the ablation occurring in real time. This allows the clinician to observe formation of ablation lesions on the ultrasound display and to immediately stop the ablation if ablation occurs in an undesired region, thereby preventing damage to healthy tissue. Further, because the system alternates between the ultrasound imaging and the ablation, the ablation does not interfere with the ultrasound imaging.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention not be limited to the details of the example embodiments.

### BRIEF DESCRIPTION OF THE FIGURES

FIG. 1 is a block diagram showing a system for visualizing formation of ablation lesions according to an embodiment of the invention.

FIG. 2 is a timing diagram showing timing for ultrasound imaging and ablation according to an embodiment of the invention.

FIG. 3 shows a system for visualizing ablation of a vagal nerve in the treatment obesity according to an embodiment of the invention.

### DETAILED DESCRIPTION

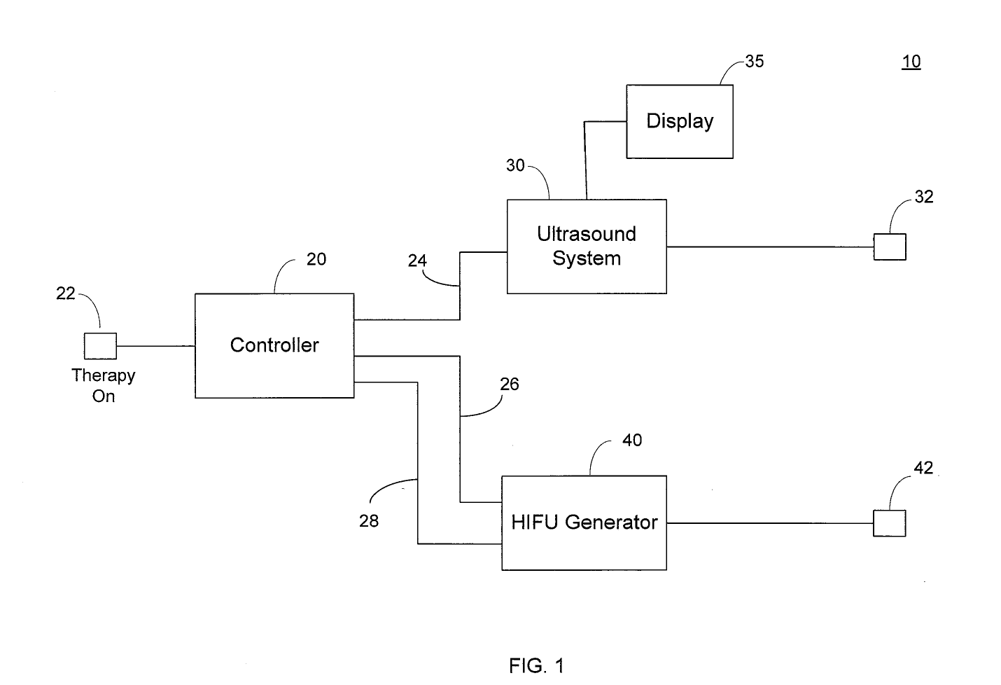

FIG. 1 shows a block diagram of a system 10 for visualizing formation of ablation lesions according to an embodiment of the invention. The system 10 includes a controller 20, an ultrasound imaging system 30, and a High Intensity Focused Ultrasound (HIFU) generator 40. The ultrasound system 30 may be a PC-based ultrasound system comprising a PC computer and an ultrasound module providing ultrasound imaging capabilities. The ultrasound system 30 is connected to an ultrasound transducer 32. The ultrasound system 30 acquires ultrasound images of the body by exciting the ultrasound transducer 32 to emit ultrasonic waves in the body. Portions of the ultrasonic waves are reflected in the body back to the transducer 32, which converts the received reflected waves into electrical signals. The electrical signal are processed by the ultrasound system 30 into ultrasound images, which are displayed on a display 35. The ultrasound transducer 32 may be mounted on a probe or catheter for acquiring ultrasound images within the body. The ultrasound system 30 includes a trigger input 24 connected to the controller 30 for triggering acquisition of an ultrasound image, as discussed further below.

The HIFU generator 40 drives an ablation transducer 42 with a high frequency signal for ablating tissue. The HIFU generator 40 receives a weak signal 26 from the controller 20, e.g., a 6 dBm signal at a frequency of 5.8-6.2 MHz. The HIFU generator 40 amplifies the weak signal 26 and drives the ablation transducer 42 with the amplified signal. To do this, the HIFU generator 40 includes a driver and a power amplifier (not shown), which are known in the art. The ablation transducer 42 may be mounted on a probe or catheter, and may be mounted on the same probe or catheter as the ultrasound transducer 32. The HIFU generator 40 includes a power control input 28 connected to the controller 30 for controlling power to the HIFU generator 40, as discussed further below.

The controller 20 controls ultrasound image acquisition by the ultrasound system 30. The controller 20 triggers the acquisition of an ultrasound image by transmitting a trigger signal (e.g., a voltage pulse) to the trigger input 24 of the ultrasound system 30. Upon receiving the trigger signal, the ultrasound system 30 acquires one ultrasound image. The controller 20 also controls power to the HIFU generator 40 through the power control input 28 of the HIFU generator 40. For example, the controller 20 may control power to the HIFU generator 40 using a switch (not shown) coupled between a power supply and the HIFU generator 40. The controller 20 also supplies the weak signal 26 to the HIFU generator 40, which the HIFU generator 40 amplifies to drive the ablation transducer 42. The controller 20 may generate the weak signal 26 using a signal synthesizer having an oscillator (not shown). The controller 20 is connected to a therapy button 22 that enables a clinician to switch ablation on and off. For example, the clinician may push the button 22 once to start ablation and release the button 22 to stop ablation. Alternatively, the controller 20 can have separate buttons for starting and stopping ablation. Alternatively or in addition to the button 22, a foot switch may be provided so that the clinician can start and stop ablation by foot.

The operation of the system 10 for visualizing formation of an ablation lesion will now be described. Before ablation, the ablation transducer 42 is positioned proximate to the tissue to be ablated. For example, the ablation transducer 42 may be on a probe that is guided to the ablation site in the body. After the ablation transducer 42 is positioned, the clinician may start ablation by pushing the therapy button 22.

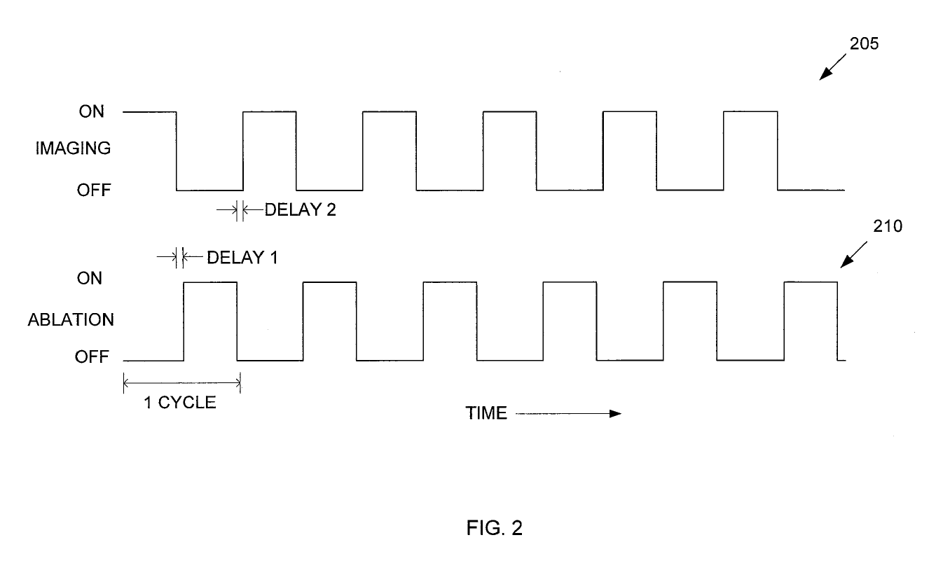

When ablation is initiated, the controller 20 alternately triggers the ultrasound system 30 to acquire an ultrasound image with the HIFU generator 40 powered off and powers on the HIFU generator 40 for a short time interval with the ultrasound imaging off. This is illustrated in the timing diagram in FIG. 2, which shows timing for the ultrasound imaging 205 and the ablation 210. During a first cycle, the controller 20 triggers the ultrasound system 30 causing the ultrasound system 30 to acquire and display an ultrasound image of the tissue being ablated. During the ultrasound image acquisition, the HIFU generator 40 is powered off. After the ultrasound system 30 is finished acquiring the ultrasound image and a short delay (Delay 1), the controller 20 powers on the HIFU generator 40 for a short time interval to ablate the tissue. After the short time interval, the HIFU generator 40 is powered off and the next cycle begins after a short delay (Delay 2). The delays are used to ensure that the ultrasound imaging and the ablation do not overlap and are optional. Table 1 shows exemplary timing parameters for a 50 ms cycle.

TABLE 1

| | |
|---|---|
| Ultrasound Imaging | 24 ms |
| Delay between Ultrasound Imaging and | 1 ms |
| Ablation | |
| HIFU powered on | 24 ms |
| Delay before next cycle | 1 ms |

In this example, the controller 20 operates at a timing frequency of 20 cycles per second. Thus, in each second, 20 ultrasound images are acquired and the HIFU generator 40 is powered on 20 separate times for 24 ms intervals.

Because the system 10 alternates between the ultrasound imaging and the ablation at a fast rate, the ultrasound system 30 appears to show the ablation occurring in real time. This allows the clinician to observe formation of ablation lesions on the ultrasound display and to immediately stop the ablation if ablation occurs in an undesired region, thereby preventing damage to healthy tissue. Further, because the system 10 alternates between the ultrasound imaging and the ablation, the ablation does not interfere with or overload the ultrasound imaging.

The timing parameters given above are exemplary only. The timing frequency can be greater than or less than 20 cycles per second. Further, the time intervals for the ultrasound imaging and/or the ablation may be adjusted. For example, the time interval for the ultrasound imaging may be adjusted according to the depth of the ultrasound images with ultrasound images at greater depths taking longer to acquire. Even though the example above used a 50-50 duty cycle between imaging on and ablation on, this need not be the case. For example, the ablation may be on for a longer time interval than the ultrasound imaging in each cycle. For example, a 25-75 duty cycle may be used in which the ablation is on three times longer than the ultrasound imaging.

Instead of triggering the ultrasound system 30 to acquire ultrasound images, the controller may control ultrasound imaging by enabling and disabling the ultrasound system 30. Further, the ultrasound transducer 32 may be part of an internal or external ultrasound imager. When ablation is not activated by the clinician, the controller 20 may continue to trigger the ultrasound system 30 to provide ultrasound imaging when the ablation is not activated. The ultrasound triggering rate when the ablation is not activated may be the same or higher than when the ablation is activated. Alternatively, the ultrasound system 30 may be taken off the triggering mode when the ablation is not activated so that the ultrasound system 30 performs ultrasound imaging without the need for external triggering.

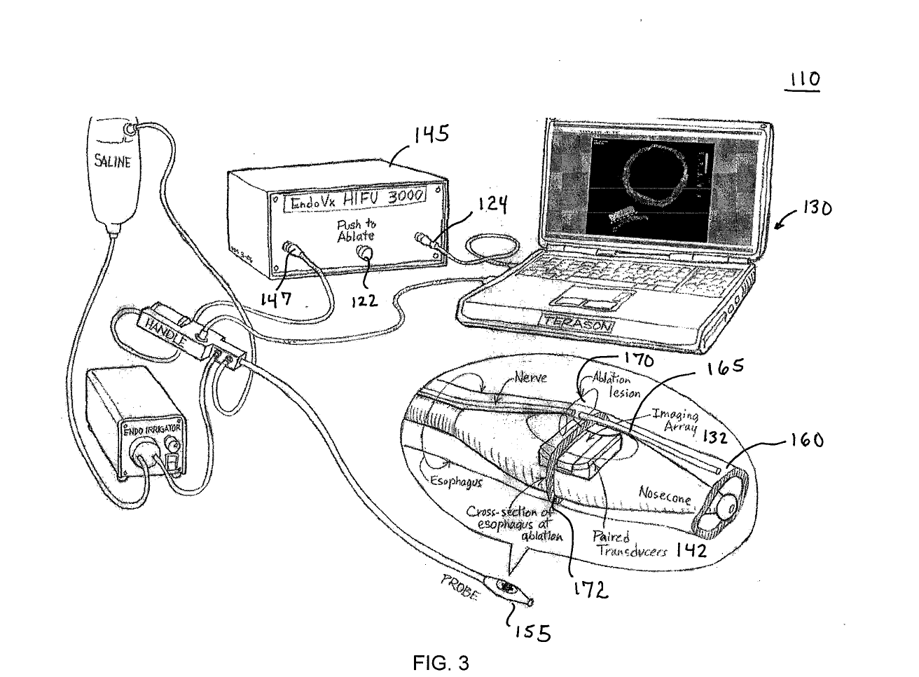

FIG. 3 shows an embodiment of the system 110, which can be used to visualize ablation of the vagal nerve in the treatment of obesity. In this embodiment, both the controller and the HIFU generator are housed in a single HIFU unit 145, and the ultrasound system is a PC-based ultrasound system 130. PC-based ultrasound systems that enable triggering of ultrasound images by an external trigger signal are commercially available from, e.g., Terason. The ultrasound transducer comprises an imaging array of ultrasound transducers 132 mounted on the distal end of an endoscopic probe 155, and the ablation transducer comprises paired transducers 142 mounted on either of the imaging array 132 on the probe 155 and configured to focus ablation energy at a desired site.

The HIFU unit 145 includes a trigger output 124 connected to the trigger input of the PC-based ultrasound system 130 for triggering ultrasound image acquisition, and an ablation signal output 147 connected to the paired transducers 142 on the probe 155 for ablation. The HIFU unit 145 also includes an ablation button 122 that enables the clinician to start and stop ablation by pushing the button 122. The PC-based ultrasound system 130 includes a ultrasound imaging module for interfacing the ultrasound imaging array 132 with the PC component of the ultrasound system 130.

Referring to the insert in FIG. 3, to ablate the vagal nerve using the system 110, a clinician guides the endoscopic probe 155 through the patient's esophagus 160 to a position in the esophagus proximate to the region of the vagal nerve 165 to be ablated. At this position, the paired transducers 142 are focused to deliver ablation energy to the vagal nerve 165 through the wall of the esophagus 172. To help focus the paired transducers 142, the ultrasound system 30 may be used to identify the position of the vagal nerve 165 relative to the paired transducers 142.

After the probe is positioned, the clinician initiates ablation by pushing the button 122. In response, the system 110 alternately acquires ultrasound images of the vagal nerve 165 and surrounding tissue using the ultrasound system 130 and ablates the vagal nerve 165 using the paired transducers 132 such that visualization of the ablation lesion 170 on the ultrasound display appears to occur in real time. This allows the clinician watching the display to quickly detect ablation in an undesired region and to stop the ablation, thereby preventing damage to the esophagus 160 and other healthy tissue surrounding the vagal nerve 165.

Ablation of the vagal nerve treats obesity by disrupting the vagal nerve. Further details on disrupting the vagal nerve to treat obesity can be found in U.S. patent application Ser. No. 10/389,236, titled “Methods and Apparatus for Treatment of Obesity,” filed Mar. 14, 2003, the entire specification of which is incorporated herein by reference.

While an embodiment of the present invention has been shown and described, various modifications may be made without departing from the scope of the present invention, and all such modifications and equivalents are intended to be covered. For example, the invention may be applied to other ablation techniques including Radio Frequency (RF) ablation. Further, the controller, HIFU generator, and ultrasound system may be housed in a single unit.

1.-25. (canceled)

26. A method for ablating a vagal nerve, comprising:
advancing an endoscopic probe through an esophagus, wherein the endoscopic probe includes an imager and an ablation device located at a distal portion of the endoscopic probe;
positioning the imager and the ablation device proximate to the vagal nerve to be ablated; and
alternately imaging the vagal nerve with the imager and ablating the vagal nerve with the ablation device at a rate of five or more cycles per second, wherein in each cycle, the imaging is performed for a first time interval and the ablating is performed for a second time interval.

26. The method of claim 26, wherein the first time interval is between approximately 24 to 100 milliseconds.

27. The method of claim 26, wherein second time interval is approximately 100 milliseconds or less.

28. The method of claim 26, wherein the ablation device comprises at least two ablation transducers, and the ablating comprises:
emitting ablation energy from each ablation transducer through a wall of the esophagus; and
focusing the ablation energy from the ablation transducers at the vagal nerve.

29. The method of claim 26, wherein in each cycle, the imaging is performed by triggering an imaging system coupled to the imager to acquire a single image, and the ablating is performed by powering on an ablation generator coupled to the ablation device.

30. The method of claim 26, wherein the imager comprises an ultrasound imager.

31. The method of claim 26, wherein the ablation device comprises a High Intensity Focused Ultrasound (HIFU) transducer.

32. The method of claim 26, wherein positioning the imager and the ablation device comprises imaging the vagal nerve and the ablation device with the imager to determine a relative position between the vagal nerve and the ablation device.

33. The method of claim 26, wherein alternately imaging and ablating the vagal nerve is performed at a rate of fifteen or more cycles per second.

34. The method of claim 26, wherein alternately imaging and ablating the vagal nerve is performed at a rate of approximately 20 cycles per second, and each of the first and second time intervals is approximately 24 milliseconds.
